(19) United States
(12) Patent Application Publication
(10) Pub. No.: US 2010/0174176 A1
(43) Pub. Date: Jul. 8, 2010

(54) MEDICAL AUXILIARY INSTRUMENT FOR INTRODUCTION INTO THE BODY OF A PATIENT

(75) Inventors: Markus Nagel, Nurnberg (DE); Ralf Petzold, Erlangen (DE)

Correspondence Address: LERNER GREENBERG STEMER LLP, P O BOX 2480, HOLLYWOOD, FL, 33022-2480, US

(73) Assignee: CAS INNOVATIONS AG, Erlangen (DE)

(21) Appl. No.: 12/377,061

(22) PCT Filed: Aug. 8, 2007
(86) PCT No.: PCT/EP2007/007013

§ 371 Date: Feb. 10, 2009

(30) Foreign Application Priority Data
Aug. 10, 2006 (DE) 10 2006 037 604.8

(51) Int. Cl. A61B 5/05

(52) U.S. Cl. 600/424

(57) ABSTRACT

A medical auxiliary instrument for introduction into the body of a patient, the use of such an auxiliary instrument, to a positioning system for percutaneous interventions, and a method for determining a reference position for such a positioning system. To permit a particularly safe minimally invasive percutaneous intervention, especially in the area of the spinal column, a medical auxiliary instrument for introduction into the body of a patient has a tube and a trocar element arranged in the tube. The trocar element has at least one marking element which is designed to determine its position with the aid of a contactless locating method. The tube, at its one end, has a securing device for forming a releasable mechanical connection to a tissue part; of the patient in such a way that, when the connection to the tissue part is established, the position of the marking element serves as a reference marker for a positioning system for percutaneous interventions.

The invention relates to a medical auxiliary instrument for introduction into the body of a patient, to the use of such an auxiliary instrument, to a positioning system for percutaneous interventions, and to a method for determining a reference position for such a positioning system.

Image-assisted interventions, in particular CT-assisted interventions, are nowadays part of clinical routine. In contrast to invasive surgical treatment, minimally invasive image-assisted interventions allow the operator to work with minimal injury to the patient. This does not just reduce the clinical costs. It also reduces the danger of complications and has a positive cosmetic effect.

To carry out these image-assisted interventions, it is necessary to navigate medical instruments through the inside of the patient's body. The term navigation is understood as determining the position by locating means, planning the access route to the target site and guiding a medical instrument to this target site along the planned access route. For this purpose, it is known to arrange electromagnetic coils both on the medical instrument and on the patient's body. When these coils are exposed to an electromagnetic field generated for this purpose, a current flow is induced in these coils. These current signals are detected and are used to determine the position of the medical instrument and thereby to navigate the latter; the coil provided on the medical instrument itself indicates the position of the medical instrument, and the coils arranged on the body of the patient serve as reference markers.

It has hitherto been customary for these reference coils to be secured, for example affixed, to the patient's skin. In complex interventions, particularly in the area of the spinal column, this type of positioning of the reference coils is too imprecise for an exact navigation of the medical instrument, however. The reason for this is that movements of the surface of the patient's body cannot be completely avoided. Reference coils therefore started to be secured directly to the patient's bone, for example using nails, clamps or screws. However, this requires invasive interventions in the patient, for which reason such a positioning of reference coils is very complex and risky and, in addition, the above-mentioned advantages of a minimally invasive intervention are for the most part negated.

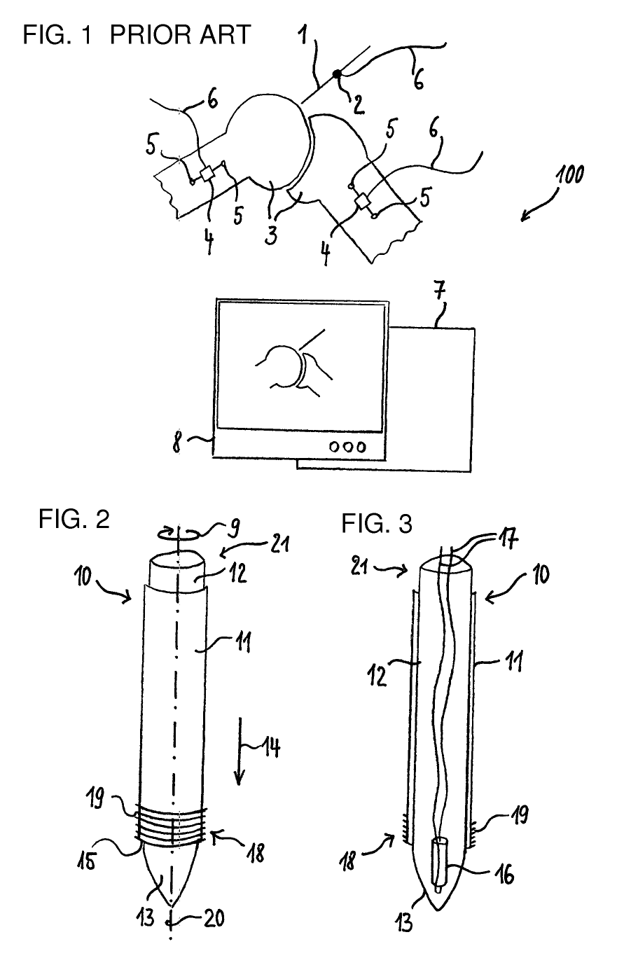

FIG. 1 shows, only by way of example, the function of a positioning system 100 known from the prior art. The medical instrument, here a biopsy needle 1, is provided with a navigation coil 2. Two reference coils 4 are secured to the bone 3 of the patient by means of screws 5. Changes in the flow of current in the coils 4 are transmitted via corresponding signal lines 6 to a central processing unit 7. Suitable data evaluation takes place there. The data are then displayed on a screen 8, for example in combination with image data from computed tomography (CT), and can be used by an operator to navigate the biopsy needle 1. Particulars concerning the structure and function of the individual components of the positioning system 100, especially the processing of the coil signals in the central processing unit 7 and the recording, that is to say collating and comparing, of the data from the coil reference system, on the one hand, and the (CT) image data, on the other hand, are known from the prior art and do not therefore have to be discussed in detail here.

It is an object of the present invention to permit a particularly safe, minimally invasive percutaneous intervention, in particular in the area of the spinal column.

This object is achieved by a medical auxiliary instrument for introduction into the body of a patient, with a tube and with a trocar element arranged in the tube, wherein the trocar element has at least one marking element which is designed such that its position can be determined by means of a contactless locating method, and wherein the tube has, at one end, a securing device for establishing a releasable mechanical connection to a tissue part of the patient in such a way that, when the connection to the tissue part is established, the position of the marking element serves as a reference marker for a positioning system for percutaneous interventions.

This object is also achieved by a positioning system for percutaneous interventions, with a medical auxiliary instrument for introduction into the body of a patient, wherein the medical auxiliary instrument has a tube and a trocar element arranged in the tube, wherein the trocar element has at least one marking element which is designed such that its position can be determined by means of a contactless locating method, and wherein the tube has, at one end, a securing device for establishing a releasable mechanical connection to a tissue part of the patient in such a way that, when the connection to the tissue part is established, the position of the marking element serves as a reference marker for the positioning system.

Finally, this object is also achieved by the use of a medical auxiliary instrument, as specified above, for providing a reference position for a positioning system for percutaneous interventions, and by a method for determining a reference position for a positioning system for percutaneous interventions, with the steps of: introducing at least one medical auxiliary instrument, as specified above, into the body of a patient, establishing a mechanical connection between the tube of the auxiliary instrument and the tissue part of the patient, and determining the position of the marking element of the auxiliary instrument by means of a contactless locating method.

A fundamental idea of the invention is for a marking element acting as reference marker to be positioned minimally invasively in the interior of the patient's body. This reference marker is preferably intended to be used for a positioning system for carrying out a computer-navigated percutaneous intervention, in particular for the navigation of a medical instrument, for example a cutting instrument or a biopsy needle, in the patient's body.

This is done using a medical auxiliary instrument which is designed in the manner of a trocar and which comprises a tube, a trocar element and a corresponding marking element. A tube is understood here as a structural part in the form of a hollow cylinder having a substantially tubular shape. The tube is preferably rigid. This permits particularly simple and safe positioning in the interior of the body. The tube can also be pliable and flexible if the trocar element is designed accordingly.

According to the invention, the marking element is brought with the auxiliary instrument to its intended site in the interior of the patient's body. In contrast to the prior art, the reference marker is arranged in the patient, and the medical instrument navigated, in a minimally invasive manner. Except for the additional openings for insertion of one or more auxiliary instruments according to the invention into the body of the patient, no other interventions are needed for fixing the reference system of the positioning system. By means of the present invention, therefore, a reference coil is placed in or directly at the target site, for example a bone tissue of the patient, without this requiring complicated interventions.

Whereas in the case of conventional biopsy needles, for example, the needle is provided exclusively for positioning the needle sleeve, i.e. basically for penetrating through the patient's skin and forming the admission channel, and is then removed again as soon as the sleeve is located at the desired site, in the present invention the positioning of the trocar element in the tube is essential to the function of the auxiliary instrument. The trocar element remains in the tube at least until the marking element has been once located.

By means of the present invention, it is possible, even with just a single auxiliary instrument according to the invention, to make available a reference system that is sufficient for safe navigation. The fixing to the bone of the patient means that no dynamic recording is necessary. After the position of the marking element has been fixed, the reference system only has to be measured again for monitoring purposes or in the event of the position of the patient having changed, for example after transport.

With an image-assisted navigation system of the kind provided according to the invention, it is possible to perform minimally invasive interventions in which even the very smallest target areas in the interior of the body can be reached safely and with precision. The invention can be used in all image-assisted interventions and treatments in which medical instruments have to be moved inside the body, particularly for the percutaneous advance of a needle to a defined anatomical position in the patient. Areas of application include biopsy procedures for removal of tissue that requires examination.

Advantageous embodiments of the invention are set forth in the dependent claims.

Particularly advantageous handling of the medical auxiliary instrument is ensured if the marking element is arranged completely within the interior of the trocar element. This means that the external diameter of the preferably pin-shaped or needle-shaped trocar element can be adapted to the internal diameter of the tube in such a way that the trocar element closes the tube substantially completely. The tube can then be more easily advanced in the tissue, and it is possible to avoid damage to the body tissue, as could occur in the case of a tube that was closed incompletely.

The marking element is preferably arranged at one end of the trocar element, this end preferably being the end of the trocar element that closes an end opening of the tube at the end of the tube comprising the securing device. This ensures that the marking element is located in or on the tissue part or at least in immediate proximity to the tissue part which is mechanically connected to the tube when the latter is fitted in place. This permits particularly exact navigation of a medical instrument relative to the tissue part, on the basis of the signals from the marking element serving as reference.

In another embodiment of the invention, the trocar element has a second marking element which is arranged at a distance from the first marking element, preferably at the opposite end of the trocar element. By using more than one marking element, it is possible to define the reference coordinate system necessary for navigation, without a further auxiliary instrument being needed to do so.

The marking elements used are preferably elements whose position can be determined by means of an electromagnetic locating method, in particular electromagnetic coils. The use of such marking elements has proven particularly safe, robust and reliable in the past. Alternatively, however, other marking elements can also be used, for example permanent magnets that serve as markers for determination of position by means of a locating method based on a purely magnetic technique.

The securing device on the tube is preferably a device designed for a releasable connection to a bone tissue, in particular a cutting thread, preferably in the form of an outer thread. However, alternative forms are possible here too, for example of such a type that a clamped connection is established instead of a screwed connection.

To ensure that the auxiliary instrument according to the invention can be used for determining a reference position in a positioning system, the auxiliary instrument is first introduced into the body of the patient. For this purpose, the trocar element can have a tip which protrudes from the front end of the tube and which ensures particularly easy introduction into the patient's body. Thereafter, the mechanical connection is established between the tube and the tissue part of the patient. In the case of a screw thread, this is done by rotating the tube, as a result of which the latter is turned into the bone of the patient. The position of the marking element is then determined, for example with the aid of the current signal of an electromagnetic coil serving as marking element in an external electromagnetic field applied for this purpose.

Illustrative embodiments of the invention are explained in more detail below with reference to the drawings, in which:

FIG. 1 shows the function of a positioning system according to the prior art,

FIG. 2 shows a medical auxiliary instrument in a top view,

FIG. 3 shows the medical auxiliary instrument from FIG. 2 in a sectional side view,

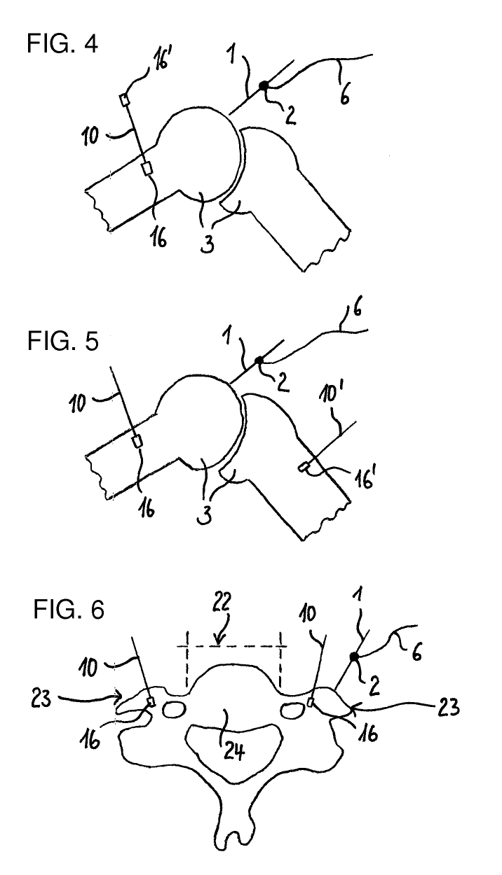

FIG. 4 shows a first use of the auxiliary instrument,

FIG. 5 shows a second use of the auxiliary instrument,

FIG. 6 shows a third use of the auxiliary instrument.

In all the figures, the invention is only shown schematically and with its principal components.

An example of a medical auxiliary instrument 10 according to the invention for introduction into the body of a patient is shown in FIGS. 2 and 3. The auxiliary instrument 10 designed in the manner of a trocar comprises a rigid tube 11, and a rigid, pin-shaped trocar element 12 arranged in the tube 11. The external diameter of the trocar element 12 is adapted to the internal diameter of the tube 11 in such a way here that the trocar element 12 closes the tube 11 substantially completely. The trocar element 12 has a tapering end 13 which closes and protrudes from the opening 15 of the tube 11 pointing in the connection or contact direction 14. The tip 13 either has a sharp cutting edge, in order to more easily produce the admission channel, or has a blunt cone shape.

A marking element in the form of an electromagnetic reference coil 16 is arranged completely within the interior of the trocar element 12. This reference coil 16 is designed in such a way that its position can be determined by means of a contactless electromagnetic locating method and thus corresponds in principle to the reference coil 4 shown in FIG. 1. For this purpose, current signals are conveyed via a signal line 17 to a central processing unit 7 of a corresponding positioning system 100 for percutaneous interventions, in order to permit navigation in the interior of the body. Instead of the reference coil, however, it is also possible to use another marking element, for example a permanent magnet, if a suitable locating method is available.

The tube 11 is open at both ends and has, at one end 18, a securing device in the form of an external cutting thread 19 for forming a releasable mechanical connection to the bone 3. In order to establish a connection to the bone 3, the auxiliary instrument 10 is pressed with its tip 13 into the bone tissue. The tube 11 is then turned in the direction of rotation 9 about its axis of rotation 20, which at the same time is its longitudinal axis, such that the cutting thread 19 is turned into the bone 3 and the tube 11 is fixed in the bone 3.

When the connection to the bone 3 has been established, the position of the reference coil 16 on or in the bone 3 serves as a reference marker for the positioning system 100.

The reference coil 16 is located in the pointed end 13 of the trocar element 12 that closes the opening 15 of the tube 11 facing in the connection direction 14. When the tip 13 has been forced into the bone 3 of the patient, a point located in immediate proximity to the bone surface is consequently used as a reference point in the reference coordinate system of the positioning system 100. This permits particularly safe navigation of the biopsy needle 1 or of another medical instrument to the bone 3.

To ensure that the reference coordinate system of the positioning system 100 can be defined unambiguously, two reference coils 16 are required. If only a single reference coil 16 were used, the position of the rotation axis 20 would be known, but not the angle position of a point remote from this axis. For this reason, a second reference coil 16′ is used whose longitudinal axis does not extend parallel to the longitudinal axis of the first reference coil 16. In this way, the angle position of a point situated remote from the rotation axis 20 can also be determined unambiguously. Evaluation of the measured data is particularly simple if the second reference coil 16′ is arranged at an angle of 90° with respect to the first reference coil 16.

The second reference coil 16′ can now be arranged as a further coil in the same auxiliary instrument 10, as is shown in FIG. 4, or it can be arranged as a reference coil 16′ in a second auxiliary instrument 10′, cf. FIG. 5. In the first case, the second reference coil 16′ is situated transversely with respect to the first reference coil 16 on the opposite end 21 of the trocar element 12 or of the tube 11 remote from the pin tip 13. In the second case, the further reference coil 16′ is once again located in the tip 13 of the trocar element 12 but, because of non-parallel placement of the further auxiliary instrument 10′ in the bone 3, it is oblique with respect to the position of the reference coil 16 of the first auxiliary instrument 10.

Applying reference coils in the area of the cervical spine has proven particularly difficult in the past. In the prior art, such coils are applied by means of clamps that are secured on the vertebral body, cf. the clamp 22 indicated by broken lines in FIG. 6. This technique is extremely complicated. It is relatively inexact and also highly risky, since a great many nerves are located in the area of the spinal column and could be damaged by such a clamp.

The use of the present invention is therefore particularly recommended in the area of the spinal column, in particular in the area of the cervical spine. Two auxiliary instruments 10, 10′ are preferably secured on the two transverse processes 23 of the vertebral body 24 (FIG. 6). This permits particularly exact referencing and, therefore, particularly safe navigation of, for example, a biopsy needle 1 to the vertebral body 24.

In contrast to a trocar in which the trocar element is removed from the tube after introduction into the body and the operating surgeon, after withdrawing the trocar element from the tube, has the possibility of introducing an optical instrument or a gripping, cutting or other medical instrument into the body, it is not necessary, for the purposes of the invention, to remove the trocar element 12 from the tube 11. However, after the reference coil 16 in the tip 13 of the trocar element 12 has been located, the trocar element 12 can be removed from the tube 11, such that the tube 11, as in a normal trocar, can then be fitted with other trocar elements, for example a biopsy needle 1.

All of the features set forth in the description, in the appended claims and in the drawing can be considered essential to the invention both individually and also in any desired combination with one another.

### LIST OF REFERENCE NUMBERS

1 biopsy needle
2 navigation coil
3 bone
4 reference coil
5 screw
6 signal line
7 central processing unit
8 screen
9 direction of rotation
10 auxiliary instrument
11 tube
12 trocar element
13 pointed end
14 direction of contact
15 tube opening
16 reference coil
17 signal line
18 tube end
19 cutting thread
20 axis of rotation
21 tube end
22 clamp
23 transverse process
24 vertebral body
100 positioning system

1-12. (canceled)

13. A medical auxiliary instrument for introduction into the body of a patient, comprising:
a tube and a trocar element disposed in said tube;
said trocar element having at least one marking element configured to enable a position thereof to be determined by way of a contactless locating method; and
said tube having an end with a securing device for establishing a releasable mechanical connection with a tissue part of the patient such that, when the connection to the tissue part is established, the position of said marking element serves as a reference marker for a positioning system for percutaneous interventions.

14. The auxiliary instrument according to claim 13, wherein said marking element is disposed completely within an interior of said trocar element.

15. The auxiliary instrument according to claim 13, wherein said marking element is disposed at one end of said trocar element.

16. The auxiliary instrument according to claim 15, wherein said marking element is disposed at that end of said trocar element that closes an end opening of said tube, at the end of said tube having said securing device.

17. The auxiliary instrument according to claim 15, wherein said marking element is a first marking element and one of said trocar element and said tube has a second marking element disposed at a distance from said first marking element.

18. The auxiliary instrument according to claim 17, wherein said second marking element is disposed at an opposite end of said trocar element or said tube.

19. The auxiliary instrument according to claim 13, wherein said marking element is configured for detecting a position thereof by way of an electromagnetic locating method.

20. The auxiliary instrument according to claim 19, wherein said marking element is an electro-magnetic coil.

21. The auxiliary instrument according to claim 13, wherein said securing device is configured for a releasable connection to bone tissue.

22. The auxiliary instrument according to claim 13, wherein said securing device is a cutting thread.

23. A positioning system for percutaneous interventions, comprising a medical auxiliary instrument according to claim 13.

24. A method of providing a reference position for a positioning system for a percutaneous intervention, which comprises providing the medical auxiliary instrument according to claim 13 and establishing the reference position with the marking element.

25. A method for determining a reference position for a positioning system for percutaneous interventions, which comprises the steps of:
introducing at least one medical auxiliary instrument according to claim 13 into the body of a patient;
establishing a mechanical connection between the tube of the auxiliary instrument and a tissue part of the patient; and
determining the position of the marking element of the auxiliary instrument by way of a contactless locating method.
